(19) United States
(12) Patent Application Publication
(10) Pub. No.: US 2024/0402202 A1
(43) Pub. Date: Dec. 5, 2024

(54) BIOLOGICAL MATERIAL CONVEYOR DEVICE

(71) Applicant: DAEGU GYEONGBUK INSTITUTE OF SCIENCE AND TECHNOLOGY, Daegu (KR)

(72) Inventors: Suk Ho PARK, Gwangju (KR); Sang Hyeon PARK, Gwangju (KR)

(21) Appl. No.: 18/718,600

(22) PCT Filed: Nov. 2, 2022
(86) PCT No.: PCT/KR2022/016960

§ 371 Date: Jun. 11, 2024

(30) Foreign Application Priority Data
Dec. 13, 2021 (KR) 10-2021-0177416

(51) Int. Cl. G01N 35/00; A61B 10/00

(52) U.S. Cl. CPC G01N 35/0098; A61B 10/0045

(57) ABSTRACT

Provided is a device for conveying biological material which can transport multiple biological materials and selectively transport the biological materials without cross-contamination. The device for conveying biological material according to one aspect of the present disclosure includes: a body part including a plurality of openings formed through the rear surface thereof, and a plurality of chambers formed therein to correspond to the openings; a plurality of transport parts accommodated in the chambers to store a biological material and capable of protruding out of the body part through the openings; and a magnetic driving part configured to be moved within the body part by an external magnetic field and to selectively apply pressure to one of the plurality of transport parts.

### TECHNICAL FIELD

The present disclosure relates to a device for conveying biological material and, more specifically, to a device for conveying biological material which can transport multiple biological materials and selectively transport the biological materials without cross-contamination.

### BACKGROUND

There are several research results showing that biological materials such as microorganisms in the digestive tract are related to not only digestive tract-related diseases (e.g., obesity, atopy, irritable bowel syndrome, inflammatory bowel disease, etc.) but also mental diseases (e.g. dementia, autism, depression, stress, anxiety disorders, etc.). Accordingly, analysis of microorganisms in the digestive tract according to location can play an important role in the diagnosis and treatment of digestive tract-related diseases or mental disorders.

In this regard, the most commonly used samples in research related to microorganisms within the digestive tract are stool samples. However, since the microbial distribution within the digestive tract varies depending on location, the stool samples have the limitation of not being able to represent the microbial distribution at a specific location within the digestive tract.

In addition, research on the role of microorganisms in the digestive tract and disease mechanisms at specific locations within the digestive tract is still insufficient.

Therefore, there is a need for a more effective method for collecting samples of biological materials such as microorganisms from various locations in the digestive tract for further research.

In addition, there is a need for a more effective method for transporting biological materials to various locations within the digestive tract to provide therapeutic effects.

In this regard, devices configured to collect and store biological materials within the digestive tract and transport specific materials into the digestive tract are being developed.

For example, as a device for collecting samples of biological material from various locations in the digestive tract, an ingestible osmotic capsule for collecting intestinal microorganisms in vivo, a capsule robot using a shape memory alloy spring for collecting intestinal microorganisms, an intestinal microorganism suction capsule, and an invasive magnetically-driven capsule for collecting intestinal microorganisms have been developed. In addition, the above-mentioned device includes a partially changed structure so as to be used to transport biological materials to various locations within the digestive tract.

However, the conventional device mentioned above only move passively, relying on peristalsis within the digestive tract and do not include an active driving function to move a device to a desired location within the digestive tract.

Especially, the conventional device cannot collect a microbial sample accurately from a desired location and does not include a multi-collection function to independently collect samples from multiple locations. In addition, the conventional device does not include a multi-transport function to individually transport multiple biological materials to multiple locations.

Therefore, there is a need for the development of a device which can transport multiple biological materials, such as microbial samples within the digestive tract, and selectively transport the biological materials without cross-contamination.

### PRIOR ART DOCUMENT(S)

Korean Paten Registration No. 10-1620624 (Published on May 24, 2016)

### DISCLOSURE

### Technical Problem

The present disclosure is to solve the above-mentioned problems involved in a device for conveying biological material.

Specifically, the present disclosure is to provide a device for conveying biological material configured to enable multiple biological materials to be transported through a multi-transport structure.

In addition, the present disclosure is to provide a device for conveying biological material configured to prevent cross-contamination between multiple biological materials being transported.

Furthermore, the present disclosure is to provide a device for conveying biological material configured to enable active movement to a desired location within the digestive tract.

The technical problems to be achieved in the present disclosure are not limited to the technical problems mentioned above, and other technical problems not mentioned will be clearly understood by those skilled in the art to which the present disclosure belongs from the description below.

### Technical Solution

In accordance with an aspect of the present disclosure, a device for conveying biological material includes: a body part including a plurality of openings formed through the rear surface thereof, and a plurality of chambers formed therein to correspond to the openings; a plurality of transport parts each accommodated in the chamber to store a biological material and capable of protruding out of the body part through the opening; and a magnetic driving part configured to be moved within the body part by an external magnetic field and to selectively apply pressure to one of the plurality of transport parts.

The body part may include a shaft disposed therein in the anteroposterior direction thereof, and the magnetic driving part may include a ring-shaped first magnetic body through which the shaft extends, so as to be movable according to the anteroposterior direction of the shaft, and a rod configured to protrude from the first magnetic body toward the chamber in which the transport part is accommodated.

The body part may include a receiving space in which the first magnetic body is disposed, the receiving space having a circular cross-section, and the first magnetic body may be rotatable around the shaft as a central axis.

The body part may further include a guide groove formed on the inner peripheral surface of the receiving space in the anteroposterior and circumferential directions thereof, and the magnetic driving part may further include a guide pin protruding from a side surface of the first magnetic body to be inserted into the guide groove.

The guide groove may include a plurality of first translation sections formed in the anteroposterior direction of the receiving space, and a rotation section formed in the circumferential direction of the receiving space to be connected to the front sides of the first translation sections, and the rotation section may be inclined in one direction between the first translation sections adjacent to each other.

In addition, the guide groove may include a plurality of first translation sections formed in the anteroposterior direction of the receiving space, a rotation section formed in the circumferential direction of the receiving space to be connected to the front sides of the first translation sections, and a plurality of second translation sections formed on the front side of the rotation section in the anteroposterior direction of the receiving space, each of the second translation sections being disposed between extension lines of the first translation sections adjacent to each other, and the rotation section may include a first inclined surface formed from the first translation section to the second translation section, and a second inclined surface formed from the second translation section to the first translation section.

The transport part may include a sealing member formed at a rear end thereof in a shape corresponding to the opening.

The transport part may further include a collection member disposed on a front side of the sealing member and capable of collecting a biological material from the subject when the transport part protrudes out of the body part.

The transport part may further include a delivery member disposed on the front side of the sealing member while storing a biological material, the delivery member being capable of delivering the biological material to the subject when the transport part protrudes out of the body part.

The transport part may further include a second magnetic body disposed at the front end thereof to generate attractive force by magnetic force with the magnetic driving part.

The magnetic driving part may be pressurized by an external magnetic field to move the body part along the lumen of the subject.

The device for conveying biological material may further include a receiving part configured to receive a signal generated from the outside of the subject by the magnetic driving part.

The front surface of the body part may have a shape including a part of a dome.

The technical problems to be achieved in the present disclosure are not limited to the technical problems mentioned above, and other technical problems not mentioned will be clearly understood by those skilled in the art to which the present disclosure belongs from the description below.

### BRIEF DESCRIPTION OF THE DRAWINGS

FIG. 1 is a perspective view of a device for conveying biological material according to an embodiment of the present disclosure.

FIG. 2 is an exploded perspective view of the device for conveying biological material according to an embodiment of the present disclosure.

FIG. 3 illustrates a state in which the device for conveying biological material according to an embodiment of the present disclosure transports a biological material.

FIG. 4 is an example of a guide groove in the device for conveying biological material according to an embodiment of the present disclosure.

FIG. 5 is a modified example of the guide groove in the device for conveying biological material according to an embodiment of the present disclosure.

FIG. 6 shows, in more detail, a magnetic driving part of the device for conveying biological material according to an embodiment of the present disclosure.

FIGS. 7 to 11 illustrate the operating states of a magnetic driving part and a transport part in the device for conveying biological material according to an embodiment of the present disclosure.

FIG. 12 shows, in more detail, the transport part of the device for conveying biological material according to an embodiment of the present disclosure.

### DETAILED DESCRIPTION

Hereinafter, preferred embodiments of the present disclosure will be described in detail with reference to the attached drawings. However, in describing the present disclosure, descriptions of already known functions or configurations will be omitted to make the gist of the present disclosure clear.

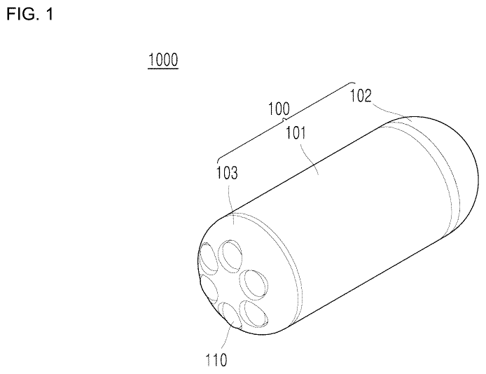

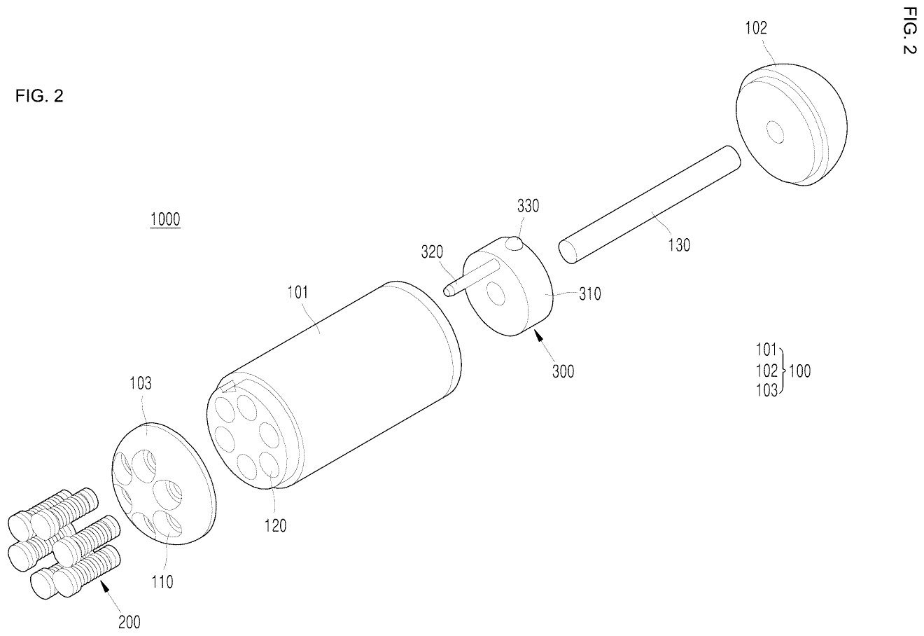

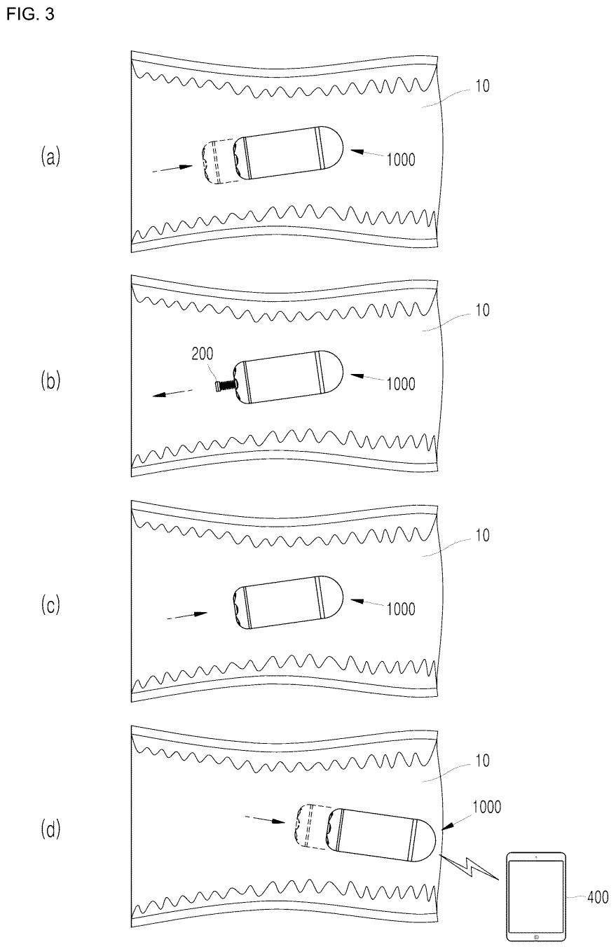

FIG. 1 is a perspective view of a device for conveying biological material according to an embodiment of the present disclosure. FIG. 2 is an exploded perspective of the device for conveying biological material according to an embodiment of the present disclosure. FIG. 3 illustrates a state in which the device for conveying biological material according to an embodiment of the present disclosure transports a biological material.

As shown in FIGS. 1 to 3, the device for conveying biological material 1000 according to an embodiment of the present disclosure includes a body part 100, a transport part 200, and a magnetic driving part 300.

In this case, the device for conveying biological material 1000 according to the present embodiment moves along the lumen of a subject 10 may store a predetermined material in a desired area of the subject 10 and transport the material or may collect a biological material from a desired area of the subject 10 and store the material, or may perform all of the mentioned.

The transport of biological material described below indicates a concept that includes i) a process of collecting a biological material from a desired area of the lumen of the subject 10 and moving the biological material in a stored state to the outside of the subject 10 and ii) a process of moving a biological material in a stored state to the lumen of the subject 10 and transporting the stored biological material to a desired area of the subject 10.

Here, the subject 10 may include an organism such as a human and an animal. In addition, the lumen of the subject 10 may include the gastrointestinal tract, the small intestine, the large intestine, etc. of the organism. Preferably, the lumen of the subject 10 refers to the large intestine.

In addition, a material delivered to the desired area of the subject 10 may include microorganisms, drugs, etc. Preferably, the material may be a probiotic microorganism.

The device for conveying biological material 1000 according to the embodiment may have a capsule shape. This shape of the device for conveying biological material 1000 may prevent the subject 10 from feeling uncomfortable about oral administration of the device for conveying biological material 1000.

The device for conveying biological material 1000 according to the embodiment may have a size suitable for moving along the lumen of the subject 10. For example, the width of the device for conveying biological material 1000 may be about 0.3 mm to about 10 mm, preferably about 1 mm to 8 mm. In addition, the length of the device for conveying biological material 1000 may be about 0.75 mm to about 30 mm, preferably about 2 mm to about 25 mm.

The device for conveying biological material 1000 according to the embodiment may employ an adhesive, such as biocompatible resin or epoxy, to seal a gap such that external fluid cannot flow in after a capsule is assembled.

The capsule as the device for conveying biological material 1000 according to the embodiment may include a fluorescent dye applied to the capsule so as to more easily identify the capsule when the capsule is recovered.

The device for conveying biological material 1000 according to the embodiment may employ coating using hydrogel, polymer, gelatin, or the like, to prevent (protect the capsule under acidic conditions and expose the capsule under neutral conditions) the capsule from being exposed in the stomach.

The device for conveying biological material 1000 according to the embodiment may employ a reagent (e.g., sterile saline solution, PBS, Quenching Reagent, etc.) along with a collection rod to prevent the death of anaerobic microorganisms, block microbial metabolic activity, and preserve dietary components in a sample.

The body part 100 is a part including a plurality of openings 110 formed through the rear surface thereof and a plurality of chambers 120 formed therein to correspond to the openings 110, and may constitute the exterior of the device for conveying biological material 1000. The body part 100 may protect the internal components of the device for conveying biological material 1000 when inserted into the lumen of the subject 10.

Particularly, the body part 100 has the opening 110 formed through the rear surface thereof with respect to the travelling direction in the lumen of the subject 10, so that the transport part 200 may protrude outside of the body part 100 through the opening 110.

In this case, the plurality of openings 110 may be provided, and a chamber 120 may be provided inside the body part 100 to correspond to each of the openings 110. The chamber 120 is a part that accommodates the transport part 200, and may have a hollow cylindrical shape, but is not necessarily limited thereto and may have various shapes according to need.

The plurality of chambers 120 are provided, and the chambers 120 are spatially separated from each other, so that the transport part 200 accommodated in each chamber 120 may perform transport independently from the transport part 200 accommodated in the chamber 120 adjacent thereto without causing contact between materials.

To this end, the body part 100 may include a plurality of partitions and a plurality of partition walls that physically separate the plurality of partitions so that the plurality of chambers 120 are formed therein (particularly, the interior of a body 101).

The body part 100 may include the body 101, a front head 102, and a rear head 103. In this case, the front head 102 may be a part formed on the front surface of the body part 100 with respect to the progress direction in the lumen of the subject 10. In addition, the rear head 103, which is a part formed on the rear surface of the body part 100 with respect to the progress direction in the lumen of the subject 10, may be a part through which the opening 110 described above is formed.

The transport part 200 is a part accommodated in each chamber 120 to store a biological material and capable of protruding out of the body part 100 through the opening 110. The plurality of transport parts 200 may be provided.

In this case, each transport part 200 may protrude out of the body part 100 such that the biological material stored in the transport part 200 inside the chamber 120 is transported to the subject 10. Alternatively, a biological material may be collected from a desired part of the subject 10 when each transport part 200 protrudes out of the body part 100, and then the biological material may be received inside the chamber 120 again together with the transport part 200.

The magnetic driving part 300 is a part which is moved within the body part 100 by an external magnetic field and selectively applies pressure to one of the plurality of transport parts 200, and may enable the transport part 200 to protrude out of the body part 100 and enable the transport part 200 to be introduced into the chamber 120 again.

To this end, a part of the magnetic driving part 300 may include a magnetic body, and the magnetic driving part 300 may perform a translational movement and/or a rotational movement within the body part 100 according to the direction in which the magnetic body is magnetized by an external magnetic field.

Accordingly, the magnetic driving part 300 may enable the transport part 200 to protrude out of the body part 100 during a translational movement in the direction of pushing the transport part 200, and the magnetic driving part 300 may enable the transport part 200 to be introduced into the chamber 120 during a translational movement in the direction of attracting the transport part 200.

In addition, when the magnetic driving part 300 rotates and is then disposed in a location at which a specific transport part 200 is pushed thereby, the magnetic driving part 300 may selectively apply pressure to one of the plurality of transport parts 200.

As described above, since the transport part 200 capable of storing a biological material is accommodated in each of the plurality of chambers 120 provided in the body part 100, the device for conveying biological material 1000 according to the embodiment can transport multiple biological materials through a multi-transport structure.

In addition, since only one of the plurality of transport parts 200 selectively protrudes out of the body part 100, the device for conveying biological material 1000 according to the embodiment can prevent cross-contamination between multiple biological materials being transported.

Referring to FIG. 3, the state in which the device for conveying biological material 1000 according to the embodiment transports a biological material is schematically described as follows.

First, as shown in (a) of FIG. 3, the device for conveying biological material 1000 may be moved to a specific location in the lumen of the subject 10. In this case, the movement of the device for conveying biological material 1000 may be steered and propelled through an external magnetic field, and the device for conveying biological material 1000 may be propelled and moved in the front direction thereof.

Then, as shown in (b) of FIG. 3, after reaching a specific location, one of the transport parts 200 may protrude out of the body part 100 by moving the magnetic driving part 300 through an external magnetic field.

In this case, a biological material may be transported to the subject 10 through the protruding transport part 200, or a material from the lumen of the subject 10 may be collected into the transport part 200.

Then, as shown in (c) of FIG. 3, after the transport or collection of the biological material is completed, the protruding transport part 200 may be inserted into the chamber 120 by moving the magnetic driving part 300 through an external magnetic field.

In this case, the chamber 120 into which the transporter part 200 has been inserted may be shielded from the other adjacent chambers 120 and the outside of the body part 100 to prevent cross-contamination of the biological material stored inside the transporter part 200.

The processes (b) and (c) of FIG. 3 may be individually performed in the plurality of transport parts 200 by repeating a process in which the magnetic driving part 300 selects a different transport part 200 to apply pressure thereto.

Next, as shown in (d) of FIG. 3, the device for conveying biological material 1000 may be moved to another location in the lumen of the subject 10 to perform the processes (b) and (c) of FIG. 3 or be discharged to the outside of the subject 10.

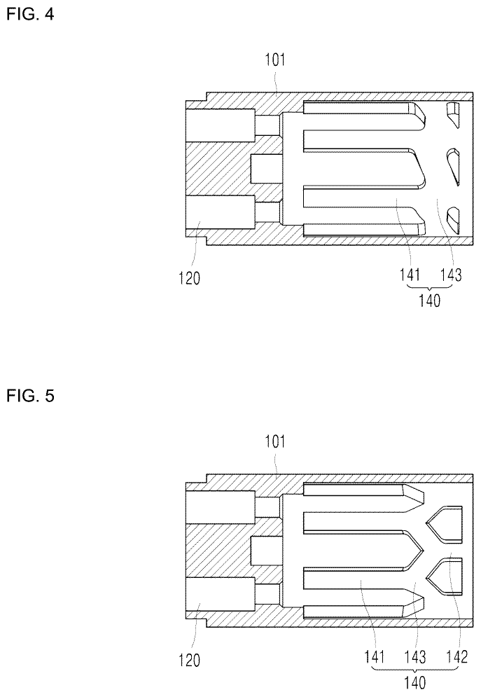

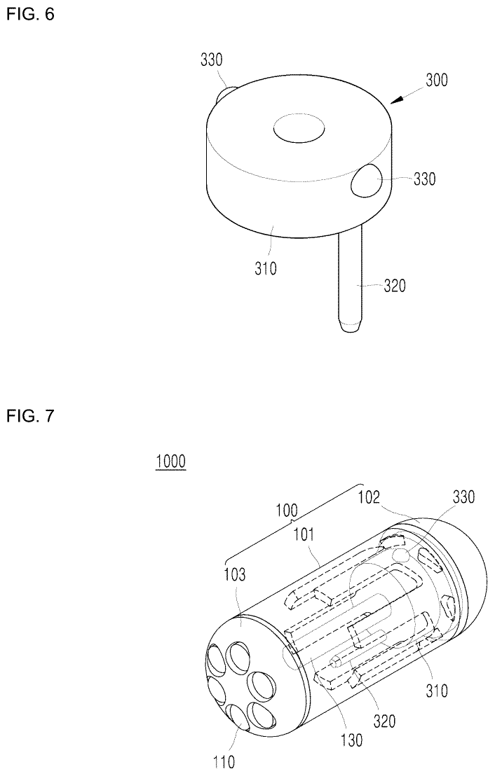

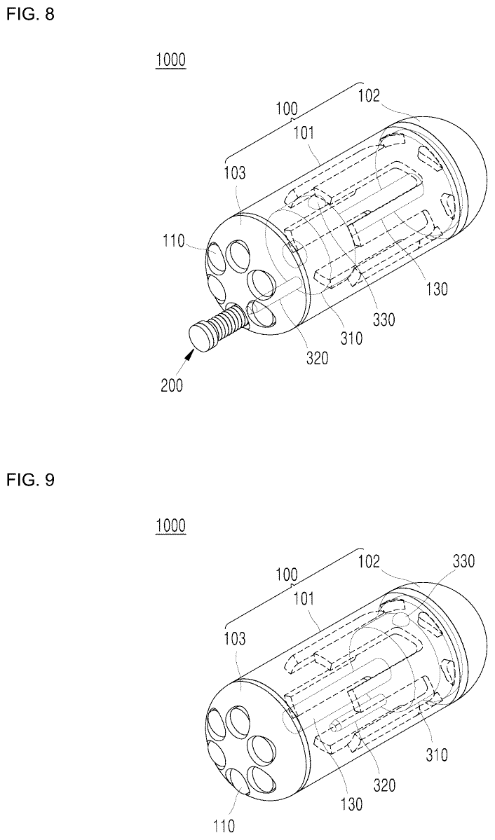

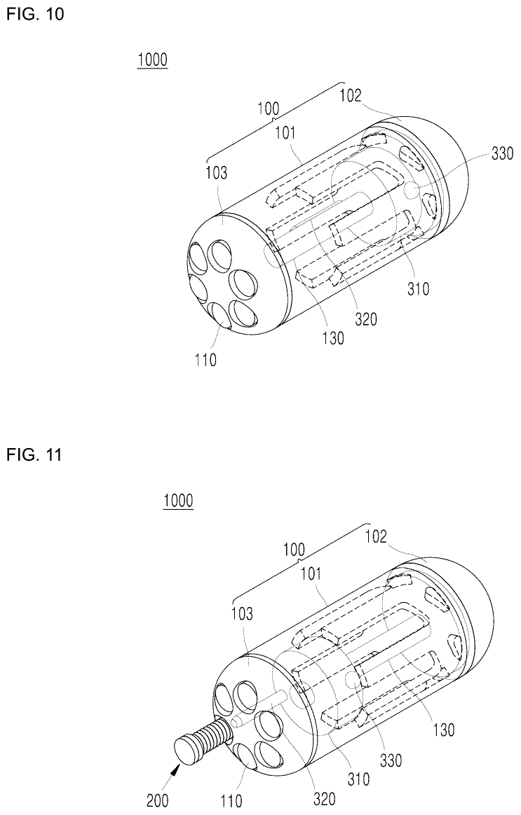

FIG. 4 is an example of a guide groove in the device for conveying biological material according to an embodiment of the present disclosure. FIG. 5 is a modified example of the guide groove in the device for conveying biological material according to an embodiment of the present disclosure. FIG. 6 shows, in more detail, a magnetic driving part of the device for conveying biological material according to an embodiment of the present disclosure. FIGS. 7 to 11 illustrate the operating states of the magnetic driving part and a transport part in the device for conveying biological material according to an embodiment of the present disclosure.

In the device for conveying biological material 1000 according to an embodiment of the present disclosure, the body part 100 may include a shaft 130 disposed therein in the anteroposterior direction. That is, the shaft 130 may be arranged according to the direction of translational movement of the magnetic driving part 300.

In this case, the magnetic driving part 300 may include a first magnetic body 310 and a rod 320.

Specifically, the first magnetic body 310 is a ring-shaped part through which the shaft 130 extends, so as to be movable according to the anteroposterior direction of the shaft 130, and may perform a translational movement within the body part 100 in the longitudinal direction of the shaft 130. In addition, the first magnetic body 310 through which the shaft 130 extends may rotate within the body part 100 around the shaft 130 as the central axis.

Particularly, since the first magnetic body 310 is magnetizable by an external magnetic field, the translational and/or rotational movements of the first magnetic body 310 may be controlled in various ways by setting the strength, frequency, and waveform of the external magnetic field differently.

The rod 320 is a part that protrudes from the first magnetic body 310 toward the chamber 120 in which the transport part 200 is accommodated, and may apply pressure to the transport part 200 accommodated in the chamber 120 during the translational movement of the first magnetic body 310.

That is, when the first magnetic body 310 performs a translational movement within the body part 100 toward the rear surface, the rod 320 may apply pressure such that the transport part 200 accommodated in the chamber 120 is pushed. In addition, when the first magnetic body 310 performs a translational movement within the body part 100 toward the front surface, the rod 320 may apply pressure such that the transport part 200 protruding out of the body part 100 is introduced.

As described above, in the device for conveying biological material 1000 according to the embodiment, the body part 100 may include the shaft 130, and the magnetic driving part 300 may include the first magnetic body 310 and the rod 320, thereby more easily and effectively pressurizing the transport part 200.

In the device for conveying biological material 1000 according to an embodiment of the present disclosure, a receiving space of the body part 100 where the first magnetic body 310 is disposed may have a circular cross-section, and the first magnetic body 310 may rotate around the shaft 130 as the center axis.

Accordingly, the magnetic driving part 300 may stably rotate within the body part 100 around the shaft 130 as the central axis, and pressurization selectively applied by the magnetic driving part 300 to one of the transport parts 200 may be performed smoothly.

In the device for conveying biological material 1000 according to an embodiment of the present disclosure, the body part 100 may further include a guide groove 140 formed on the inner peripheral surface of the receiving space in the anteroposterior and circumferential directions thereof.

In this case, the magnetic driving part 300 may further include a guide pin 330 protruding from the side surface of the first magnetic body 310 to be inserted into the guide groove 140.

That is, the magnetic driving part 300 may be disposed inside the body part 100 such that the guide pin 330 protruding from the side surface of the first magnetic body 310 is inserted into the guide groove 140 of the body part 100. In addition, when the magnetic driving part 300 performs a translational movement and/or rotational movement due to an external magnetic field, the guide pin 330 inserted into the guide groove 140 may move in the direction in which the guide groove 140 is formed.

Therefore, the movement direction of the magnetic driving part 300 may be standardized and stably managed during the control of the magnetic driving part 300 through an external magnetic field, thereby more easily controlling the device for conveying biological material 1000 according to the embodiment.

The guide pins 330 may be formed in a pair on opposite sides of the first magnetic body 310, but are not necessarily limited thereto, and the number and location of the guide pins 330 may vary as needed.

In the device for conveying biological material 1000 according to an embodiment of the present disclosure, the guide groove 140 may include a plurality of first translation sections 141 formed in the anteroposterior direction of the receiving space, and a rotation section 143 formed in the circumferential direction of the receiving space to be connected to the front sides of the first translation sections 141. In this case, the rotation section 143 may be inclined in one direction between the adjacent first translation sections 141.

That is, as shown in FIG. 4, the first translation section 141 of the guide groove 140 may be formed in the anteroposterior direction of the body part 100. Accordingly, the magnetic driving part 300 and the guide pin 330 inserted into the first translation section 141 may perform a translational movement in the anteroposterior direction of the body part 100.

In addition, the rotation section 143 of the guide groove 140 may be formed in the circumferential direction of the body part 100. Accordingly, the magnetic driving part 300 and the guide pin 330 inserted into the rotation section 143 may rotate in the circumferential direction of the body part 100.

Particularly, since the rotation section 143 is inclined in one direction between adjacent first translation sections 141, the guide pin 330 moved from one of the first translation sections 141 to the rotation section 143 may be naturally moved to another adjacent first translation section 141 along the inclined surface.

That is, since the rotational movement of the magnetic driving part 300 is naturally performed in stages during the translational movement of the magnetic driving part 300, pressurization selectively applied to any one of the plurality of transport parts 200 may be sequentially performed.

In this case, if a certain amount of rotational force is applied during the translational movement of the magnetic driving part 300 through an external magnetic field to the magnetic driving part 300 through the external magnetic field, the rotational force may have further effect on the mechanism for naturally moving the guide pin 330 to the adjacent translation section 141 along the inclined surface, as described above.

In the device for conveying biological material 1000 according to an embodiment of the present disclosure, the guide groove 140 may include a plurality of first translation section 141 formed in the anteroposterior direction of the receiving space, a rotation section 143 formed in the circumferential direction of the receiving space to be connected to the front sides of the first translation sections 141, and a plurality of second translation sections 142 formed on the front side of the rotation section 143 in the anteroposterior direction of the receiving space and respectively disposed between the extension lines of the adjacent first translation sections 141. In this case, the rotation section 143 may include a first inclined surface formed from the first translation section 141 to the second translation section 142, and a second inclined surface formed from the second translation section 142 to the first translation section 141.

That is, as shown in FIG. 5, the first translation section 141 of the guide groove 140 may be formed in the anteroposterior direction of the body part 100. In addition, the rotation section 143 of the guide groove 140 may be formed in the circumferential direction of the body part 100.

The second translation section 142 of the guide groove 140 may also be formed in the anteroposterior direction of the body part 100 like the first translation section 141, but the second translation section 142 may be misaligned with the first translation section 141.

Particularly, since the rotation section 143 has the first inclined surface and the second inclined surface formed between the first translation section 141 and the second translation section 142, which are adjacent to each other, the guide pin 330 moved from one of the first translation sections 141 to the rotation section 143 may be naturally moved to an adjacent second translation section 142 along the first inclined surface.

The guide pin 330, which has moved from the second translation section 142 back to the rotation section 143, may naturally move to another first translation section 141 along the second inclined surface.

That is, since the rotational movement of the magnetic driving part 300 is naturally performed in stages during the translational movement of the magnetic driving part 300, pressurization selectively applied to any one of the plurality of transport parts 200 may be sequentially performed.

In this case as well, if a certain amount of rotational force is applied during the translational movement of the magnetic driving part 300 through an external magnetic field to the magnetic driving part 300 through the external magnetic field, it may be further effective in the mechanism for naturally moving the guide pin 330 to the adjacent translation section 141 along the first inclined surface and the second inclined surface, as described above.

Referring to FIGS. 7 to 11, the operating states of the magnetic driving part 300 and the transport part 200 of the device for conveying biological material 1000 according to the embodiment are schematically described as follows.

First, as shown in FIG. 7, the rod 320 of the magnetic driving part 300 may be aligned with the transport part 200 that is to be protruded out of the body part 100. In this case, the rod 320 may be aligned by rotating the first magnetic body 310 through an external magnetic field.

Then, as shown in FIG. 8, the magnetic driving part 300 may be translated in the longitudinal direction of the shaft 130 through an external magnetic field to push the transport part 200 with which the rod 320 is aligned.

In this case, since the guide pin 330 of the magnetic driving part 300 is inserted into the first translation section 141 of the guide groove 140, the magnetic driving part 300 may perform a translational movement toward the rear surface of the body part 100 without a separate rotational movement.

Then, as shown in FIG. 9, the magnetic driving part 300 may be translated in the longitudinal direction of the shaft 130 through an external magnetic field to attract the transport part 200 with which the rod 320 is aligned.

In this case as well, since the guide pin 330 of the magnetic driving part 300 is inserted into the first translation section 141 of the guide groove 140, the magnetic driving part 300 may perform a translational movement toward the front surface of the body part 100 without any separate rotational movement.

Then, as shown in FIG. 10, when the guide pin 330 of the magnetic driving part 300 reaches the rotation section 143 of the guide groove 140, the magnetic driving part 300 may naturally move along the inclined surface, and accordingly, perform a rotational movement.

Accordingly, the rod 320 of the magnetic driving part 300 may be aligned with another transport part 200 adjacent to the protruding transport part 200 in FIG. 8.

Then, as shown in FIG. 11, the magnetic driving part 300 may be translated in the longitudinal direction of the shaft 130 through an external magnetic field such that the rod 320 pushes another transport part 200 adjacent to the protruding transport part 200 in FIG. 8.

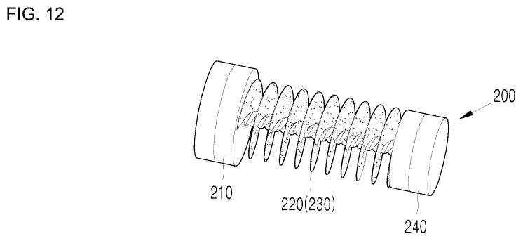

FIG. 12 shows, in more detail, the transport part of the device for conveying biological material according to an embodiment of the present disclosure.

In the device for conveying biological material 1000 according to an embodiment of the present disclosure, the transport part 200 may include a sealing member 210 formed at the rear end thereof in a shape corresponding to the opening 110.

As described above, when the transport part 200 transports a biological material to the subject 10 or collects a biological material from the subject 10, the chamber 120 in which the transport part 200 is accommodated needs to be sealed from the outside.

Therefore, as shown in FIG. 12, the sealing member 210 may be disposed at the rear end of the transport part 200 so that the sealing member 210 seals the opening 110 while the transport part 200 is accommodated in the chamber 120.

In this case, the sealing member 210 may include rubber or the like formed in a shape corresponding to the opening 110, but is not necessarily limited thereto, and may include various materials as needed.

In the device for conveying biological material 1000 according to an embodiment of the present disclosure, the transport part 200 may further include a collection member 220 disposed on the front side of the sealing member 210 and capable of collecting a biological material from the subject 10 when the transport part 200 protrudes out of the body part 100.

That is, the collection member 220 may be configured to collect a biological material from a desired area of the subject 10, and may include a sorption element to which a biological material is sorbed. For example, the sorption element may include a sponge, polystyrene made of Dacron, and the like.

In the device for conveying biological material 1000 according to an embodiment of the present disclosure, the transport part 200 may further include a delivery member 230 disposed on the front side of the sealing member 210 while storing a biological material, the delivery member being capable of delivering the biological material to the subject 10 when the transport part 200 protrudes out of the body part 100.

That is, the delivery member 230 may be configured to be accommodated within the chamber 120 while storing a biological material, and may be configured to release the biological material when protruding out of the body part 100.

For example, the delivery member 230 may accommodate a capsule including at least one biological material containing a material to be delivered to a desired area of the subject 10. In this case, the capsule may be formed of a suitable material when decomposed in the lumen of the subject 10.

The capsule may be formed of a material that does not decompose in the stomach in order to pass intactly through the small or large intestine of the subject 10 but decomposes in the small or large intestine. In this case, the capsule may be coated with an enteric coating, a chitosan coating, etc.

In the device for conveying biological material 1000 according to an embodiment of the present disclosure, the transport part 200 may further include a second magnetic body 240 disposed at the front end thereof to generate attractive force by magnetic force with the magnetic driving part 300.

That is, as shown in FIG. 12, the second magnetic body 240 may be disposed at a part in contact with the rod 320 of the magnetic driving part 300.

As described above, when the magnetic driving part 300 attracts the protruding transport part 200 back into the chamber 120 while performing a translational movement, physical bonding may be required between the magnetic driving part 300 (particularly, the rod 320) and the transport part 200.

In this case, the overall structure may be complicated in that a separate fastening and separation structure needs to be applied.

Accordingly, the second magnetic body 240 may be disposed at the front end of the transport part 200 so that the protruding transport part 200 is easily drawn into the chamber 120 without a separate fastening and separation structure.

Accordingly, the rod 320 of the magnetic driving part 300 and the second magnetic body 240 may be attracted to each other through magnetic force, and the transport part 200 may be attracted together in the moving direction of the magnetic driving part 300 by attractive force during the translational movement of the magnetic driving part 300.

The second magnetic body 240 may be made of a material including an elastic member. As described above, while the rod 320 of the magnetic driving part 300 pressurizes the transport part 200, a predetermined impact may be applied to the second magnetic body 240 in contact with the rod 320.

Particularly, while the rod 320 of the magnetic driving part 300 pushes the transporter part 200, significant pressure may be applied to the second magnetic body 240, and thus, the impact and vibration resulting therefrom need to be offset.

Therefore, the second magnetic body 240 may be formed of an elastic material such as a rubber magnet, thereby performing a buffering function between members while generating attractive force by means of magnetic force.

In addition, the second magnetic body 240 may be formed of an elastic material such as a rubber magnet to enable a sealing function to be partially achieved even at the front end of the transport part 200, thereby more effectively preventing cross-contamination from occurring in the plurality of chambers.

In the device for conveying biological material 1000 according to an embodiment of the present disclosure, the magnetic driving part 300 may be pressurized by an external magnetic field to move the body part 100 along the lumen of the subject 10.

That is, the body part 100 may be moved by pressurizing the magnetic driving part 300 by an external magnetic field, without a separate driving part for moving the body part 100.

As described above, in the device for conveying biological material 1000 according to the embodiment, the magnetic driving part 300 pressurized by an external magnetic field moves the body part 100 along the lumen of the subject 10, and therefore, the device for conveying biological material 1000 can be actively moved to a desired location within the digestive tract.

In order that the device for conveying biological material 1000 according to the embodiment collects a biological material from the lumen of the subject 10, a specific transport part 200 protruding to the outside of the body part 100 needs to be in contact with the bottom surface of the lumen of the subject 10.

Accordingly, the device for conveying biological material 1000 may be controlled through an external magnetic field such that a specific opening 100 among the plurality of openings 110, corresponding to the transport part 200 that is to be protruded to the outside of the body part 100, is aligned toward the bottom surface of the lumen of the subject 10.

The device for conveying biological material 1000 according to the embodiment may include an electric energy storage element, a communication element, and a control element.

The electric energy storage element may supply electric energy to the device for conveying biological material 1000. For example, the electric energy storage element may include a battery.

The communication element may communicate with an external device of the device for conveying biological material 1000. The communication element may include a signal generator configured to generate an externally recognizable location signal. The location of the device for conveying biological material 1000 may be detected based on the location signal generated from the signal generator.

For example, the communication element may generate a radio frequency signal (RF signal). As another example, the communication element may generate a magnetic field. The communication element may be implemented as an antenna, a permanent magnet, an electromagnet, etc.

The control element may control the electric energy storage element, the communication element, etc. For example, the control element may include a circuit board.

In addition, the device for conveying biological material 1000 according to the embodiment may be detected in the lumen of the subject 10 by an external signal. For example, the location of the device for conveying biological material 1000 in the lumen of the subject 10 may be detected using external X-rays.

Alternatively, the location of the device for conveying biological material 1000 in the lumen of the subject 10 may be detected using external ultrasound waves.

Alternatively, as an RF or magnetic field method, a permanent magnet or electromagnet capable of generating an RF signal or magnetic field may be built inside the device for conveying biological material 1000, thereby detecting the location of the device for conveying biological material 1000 in the lumen of the subject by using an external receiver (RF receiver or magnetic field sensor).

In this regard, the device for conveying biological material 1000 according to an embodiment of the present disclosure may further include a receiving part 400 configured to receive a signal generated from the outside of the subject 10 by the magnetic driving part 300.

The device for conveying biological material 1000 according to the embodiment may measure the magnetic field by using a plurality of 3-axis magnetometers and may estimate the location and posture of the device for conveying biological material 1000 by using the measured values.

In the device for conveying biological material 1000 according to an embodiment of the present disclosure, the front surface of the body part 100 may have a shape including a part of a dome. That is, as shown in FIGS. 1 and 2, the front head 102 of the body part 100 may have a shape including a part of a dome.

The body part 100 may receive resistance according to a material on the lumen of the subject 10 when moving along the lumen of the subject 10. Accordingly, the front surface of the body part 100 may have a streamlined shape or a dome shape.

Although specific embodiments of the present disclosure have been described and shown above, the present disclosure is not limited to the described embodiments, and it is obvious to those skilled in the art that various modifications and changes can be made without departing from the spirit and scope of the present disclosure. Accordingly, such modifications or variations should not be understood individually from the technical idea or viewpoint of the present disclosure, and the modified embodiments should be regarded as falling within the scope of the claims of the present disclosure.

Descriptions of the Reference Numerals

| | |
|---|---|
| 10: Subject | 100: Body part |
| 101: Body | 102: Front head |
| 103: Rear head | 110: Opening |
| 120: Chamber | 130: Shaft |
| 140: Guide groove | 141: First translation section |
| 142: Second translation section | 143: Rotation section |
| 200: Transport part | 210: Sealing member |
| 220: Collection member | 230: Delivery member |
| 240: Second magnetic body | 300: Magnetic driving part |
| 310: First magnetic body | 320: Rod |
| 330: Guide pin | 400: Receiving part |
| 1000: Device for conveying | |
| biological material | |

### INDUSTRIAL APPLICABILITY

According to at least one of the embodiments of the present disclosure, the transport part capable of storing a biological material may be accommodated in each of the plurality of chambers formed in the body part, thereby transporting multiple biological materials through the multi-transport structure.

In addition, according to at least one of the embodiments of the present disclosure, only one of the plurality of transport parts may selectively protrude out of the body part, thereby preventing cross-contamination between multiple biological materials being transported.

Furthermore, according to at least one of the embodiments of the present disclosure, the magnetic driving part pressurized by an external magnetic field may move the body part along the lumen of the subject, thereby actively moving the device for conveying biological material to a desired location within the digestive tract.

1. A device for conveying biological material comprising:
a body part comprising a plurality of openings formed through a rear surface thereof, and a plurality of chambers formed therein to correspond to the openings;
a plurality of transport parts each accommodated in the chamber to store a biological material and capable of protruding out of the body part through the opening; and
a magnetic driving part configured to be moved within the body part by an external magnetic field and to selectively apply pressure to one of the plurality of transport parts.

2. The device for conveying biological material of claim 1,
wherein the body part comprises a shaft disposed therein in the anteroposterior direction thereof, and
wherein the magnetic driving part comprises:
a ring-shaped first magnetic body through which the shaft extends, so as to be movable according to the anteroposterior direction of the shaft; and
a rod configured to protrude from the first magnetic body toward the chamber in which the transport part is accommodated.

3. The device for conveying biological material of claim 2,
wherein the body part comprises a receiving space in which the first magnetic body is disposed, the receiving space having a circular cross-section, and
wherein the first magnetic body is rotatable around the shaft as a central axis.

4. The device for conveying biological material of claim 3,
wherein the body part further comprises a guide groove formed on the inner peripheral surface of the receiving space in the anteroposterior and circumferential directions thereof, and
wherein the magnetic driving part further comprises a guide pin protruding from a side surface of the first magnetic body to be inserted into the guide groove.

5. The device for conveying biological material of claim 4,
wherein the guide groove comprises:
a plurality of first translation sections formed in the anteroposterior direction of the receiving space; and
a rotation section formed in the circumferential direction of the receiving space to be connected to the front sides of the first translation sections, and
wherein the rotation section is inclined in one direction between the first translation sections adjacent to each other.

6. The device for conveying biological material of claim 4,
wherein the guide groove comprises:
a plurality of first translation sections formed in the anteroposterior direction of the receiving space;
a rotation section formed in the circumferential direction of the receiving space to be connected to the front sides of the first translation sections; and
a plurality of second translation sections formed on the front side of the rotation section in the anteroposterior direction of the receiving space, each of the second translation sections being disposed between extension lines of the first translation sections adjacent to each other, and
wherein the rotation section comprises a first inclined surface formed from the first translation section to the second translation section, and a second inclined surface formed from the second translation section to the first translation section.

7. The device for conveying biological material of claim 1,
wherein the transport part comprises a sealing member formed at a rear end thereof in a shape corresponding to the opening.

8. The device for conveying biological material of claim 7,
wherein the transport part further comprises a collection member disposed on a front side of the sealing member and capable of collecting a biological material from a subject when the transport part protrudes out of the body part.

9. The device for conveying biological material of claim 7,
wherein the transport part further comprises a delivery member disposed on the front side of the sealing member while storing a biological material, the delivery member being capable of delivering the biological material to the subject when the transport part protrudes out of the body part.

10. The device for conveying biological material of claim 7,
wherein the transport part further comprises a second magnetic body disposed at a front end thereof to generate attractive force by magnetic force with the magnetic driving part.

11. The device for conveying biological material of claim 1,
wherein the magnetic driving part is pressurized by an external magnetic field to move the body part along the lumen of the subject.

12. The device for conveying biological material of claim 11,
further comprising a receiving part configured to receive a signal generated from the outside of the subject by the magnetic driving part.

13. The device for conveying biological material of claim 11,
wherein the front surface of the body part partially comprises a dome shape.
